(19) United States
(12) Patent Application Publication
(10) Pub. No.: US 2026/0096857 A1
(43) Pub. Date: Apr. 9, 2026

(54) METHOD FOR USING FIDUCIAL MARKER BY USING ANATOMICAL STRUCTURE IN SURGICAL NAVIGATION SYSTEM

(71) Applicant: DECASIGHT CORPORATION, Seoul (KR)

(72) Inventors: Byung Joon PARK, Seoul (KR); Soo Ho CHOI, Ansan-si, Gyeonggi-do (KR); Tae Hyun KIM, Seoul (KR); Ji Ho PARK, Seoul (KR); Ki Wook LEE, Incheon (KR)

(21) Appl. No.: 19/114,161

(22) PCT Filed: Apr. 6, 2023
(86) PCT No.: PCT/KR2023/004636

§ 371 Date: Mar. 21, 2025

(30) Foreign Application Priority Data
Mar. 24, 2023 (KR) 10-2023-0038709

(51) Int. Cl. A61B 34/20; A61B 34/10; A61B 90/00; G06T 7/33; G06T 17/00; G06T 19/20; G06V 10/26

(52) U.S. Cl. CPC A61B 34/20; A61B 90/39; G06T 7/344; G06T 17/00; G06T 19/20; G06V 10/26; A61B 2034/105; A61B 2034/2065; A61B 2090/373; A61B 2090/3762; A61B 2090/3933; A61B 2090/3966; A61B 2090/3979; G06T 2207/10064; G06T 2207/10081; G06T 2207/30204; G06T 2210/41; G06T 2219/2004; G06V 2201/03

(57) ABSTRACT

The present invention relates to a method for using a fiducial marker in a surgical navigation system, comprising: a computed tomography (CT) process step of obtaining a CT image through CT scanning of a patient; a CT image-based 3D reconstruction step of generating, on the basis of the CT image, a CT 3D model by using 3D reconstruction technology; a fluorescence process step of generating a fluorescent image by capturing, using a multi-camera, an anatomical landmark in which an anatomical structure near a surgical target of the patient is highlighted with a fluorescent contrast agent; a multi-camera-based 3D reconstruction step of generating a fluorescent 3D model from the fluorescent image by using the 3D reconstruction technology; and a registration step of registering the CT 3D model and the fluorescent 3D model into one model.

### TECHNICAL FIELD

The present disclosure relates to a surgical navigation system and, more specifically, to a method of using a fiducial marker in a surgical navigation system.

### BACKGROUND ART

In a recent medical field, surgical navigation is used for more precise and safer surgeries. To use this surgical navigation, fiducial markers should be utilized. A fiducial marker refers to a marker that serves as a reference in medical imaging and other applications.

In the existing surgical navigation, a fiducial marker is installed on the patient for use. The fiducial marker used here may be classified into invasive and non-invasive types.

FIG. 1 illustrates a non-invasive fiducial marker, and FIG. 2 illustrates an invasive fiducial marker.

Referring to FIGS. 1 and 2, a non-invasive fiducial marker is attached to the skin, thus placing no burden on the patient. However, the non-invasive fiducial marker cannot be securely fixed due to the mobility of the skin, which introduces potential errors.

In contrast, an invasive fiducial marker is securely fixed to a patient while ensuring stability. However, the invasive fiducial marker should be attached to hard tissues such as bones in the body, thus placing a burden on the patient.

Commonly, both the non-invasive method and the invasive method involve a large distance between a surgical target and a fiducial marker, which may increase the target registration error (TRE) and reduce accuracy.

FIG. 3 is an illustrative diagram for explaining FLE, FRE, and TRE.

Referring to FIG. 3, FLE is an abbreviation for fiducial localization error, which means the error between each actual fiducial point and each measured point.

FRE is an abbreviation for fiducial registration error, which refers to the error of the fiducial point in two spaces measured after registration. That is, FRE refers to the error between each aligned fiducial point.

TRE is an abbreviation for target registration error, which refers to the error between an actual surgical target point and a surgical target point in a registered model.

When the distance between the fiducial marker set and the surgical target is large, the TRE increases due to the distance between the surgical target and the fiducial marker even if the error of FRE is small. In other words, the smaller the distance between the fiducial marker set and the surgical target, the smaller the TRE value.

Accordingly, in the case of fiducial markers installed on patients for surgical navigation, the physical burden or stability as a guidance indicator may decrease depending on the type of marker used.

### DISCLOSURE

### Technical Problem

The present disclosure has been made to solve the problems above and is to provide a method of using an anatomical structure near a surgical target as a fiducial marker in a surgical navigation system, by using a fluorescent contrast agent together with a non-invasive fiducial marker.

The present disclosure is not limited to the foregoing, and other aspects not mentioned will be clearly understood by those skilled in the art from the description below.

### Technical Solution

In view of the foregoing, the present disclosure relates to a method of using a fiducial marker in a surgical navigation system, including a computed tomography (CT) process for obtaining a CT image via a CT scan of a patient, CT image-based 3D reconstruction for generating, based on the CT image, a CT 3D model by using 3D reconstruction technology, a fluorescence process for generating a fluorescent image by capturing, using multiple cameras, an image of an anatomical landmark in which an anatomical structure near a surgical target of the patient is highlighted with a fluorescent contrast agent, multi-camera-based 3D reconstruction for generating a fluorescent 3D model from the fluorescent image by using the 3D reconstruction technology, and registration for registering the CT 3D model and the fluorescent 3D model into one model.

The CT process may include performing a computed tomography (CT) scan of the patient including the fiducial marker attached to the patient's skin, and segmenting a CT image captured via the CT scan, by using a selected surgical target and anatomical structure of the patient.

In the CT image-based 3D reconstruction, the CT image may be reconstructed into a 3D model by using marching cube algorithm-based 3D reconstruction technology.

The fluorescence process may include waiting until the fluorescent contrast agent injected to target the anatomical structure is deposited, and capturing an image by using multiple near-infrared cameras when the fluorescent contrast agent is deposited.

The registration may include coarse registration of registering the CT 3D model and the fluorescent 3D model into one model, and fine registration of more precisely registering the model obtained after performing the coarse registration.

### Advantageous Effects

The present disclosure can reduce the physical burden on a patient and improve surgical precision, by using an anatomical structure as a reference marker in a surgical navigation system.

### DESCRIPTION OF DRAWINGS

FIG. 1 illustrates a non-invasive fiducial marker.

FIG. 2 illustrates an invasive fiducial marker.

FIG. 3 is an illustrative diagram for explaining FLE, FRE, and TRE.

FIG. 4 is a flow chart showing a method of using a fiducial marker using an anatomical structure in a surgical navigation system according to an embodiment of the present disclosure.

FIG. 5 illustrates 3D reconstruction of a CT image according to an embodiment of the present disclosure.

FIG. 6 is a flow chart showing a CT 3D reconstruction process according to an embodiment of the present disclosure.

FIG. 7 illustrates a fluorescent 3D reconstruction process according to an embodiment of the present disclosure.

FIG. 8 is a flow chart showing a fluorescent 3D reconstruction process according to an embodiment of the present disclosure.

FIG. 9 is a photograph obtained by using a fluorescent contrast agent.

FIG. 10 illustrates a coarse registration process according to an embodiment of the present disclosure.

FIG. 11 illustrates a registration process to which an ICP algorithm is applied.

FIG. 12 shows images before and after an ICP algorithm technology is applied.

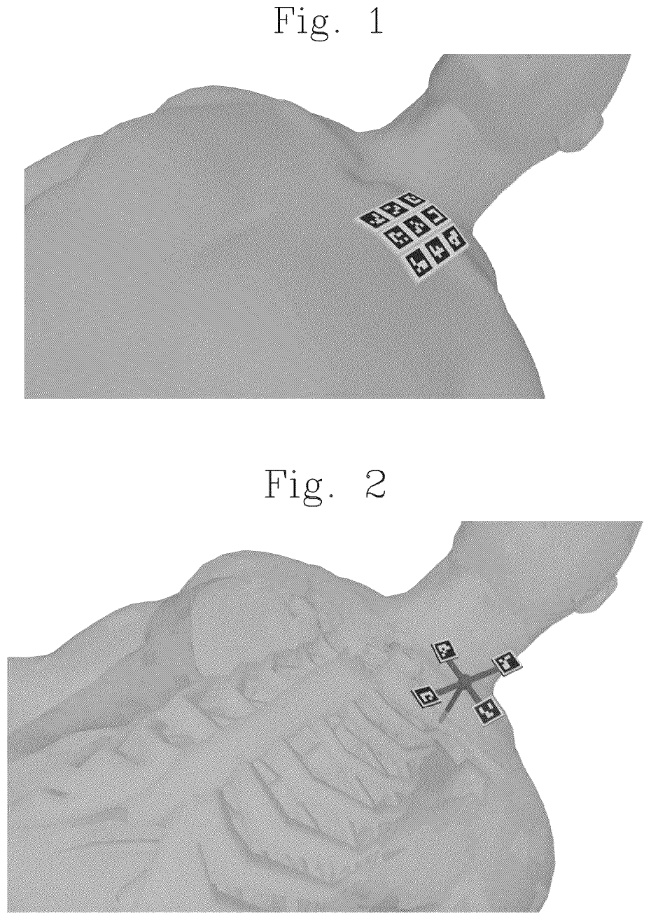

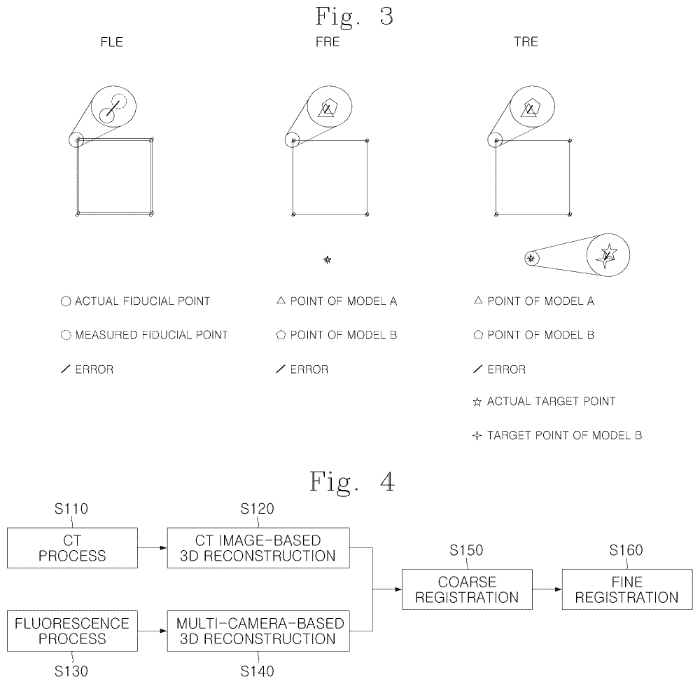

### MODE FOR INVENTION

FIG. 4 is a flow chart showing a method of using a fiducial marker using an anatomical structure in a surgical navigation system according to an embodiment of the present disclosure.

Referring to FIG. 4, the method includes a CT process (S110) of acquiring a CT image via a computed tomography (CT) scan of a patient, CT image-based 3D reconstruction (S120) of generating a CT 3D model by using 3D reconstruction technology, based on the CT image, a fluorescence process (S130) of generating a fluorescent image by capturing, using multiple cameras, an image of an anatomical landmark in which an anatomical structure near a surgical target of the patient is highlighted with a fluorescent contrast agent, multi-camera-based 3D reconstruction (S140) of generating a fluorescent 3D model from the fluorescent image by using 3D reconstruction technology, coarse registration (S150) of registering the CT 3D model and the fluorescent 3D model into one model, and fine registration (S160) of more precisely registering the model obtained after performing the coarse registration.

The present disclosure is subject to various modifications and may have various embodiments, and specific embodiments are illustrated in the drawings and described in detail. However, this is not intended to limit the present disclosure to specific embodiments, and it should be understood to include all modifications, equivalents, and substitutes included in the spirit and technical scope of the present disclosure.

The terms used in this application are used only to describe specific embodiments and are not intended to limit the present disclosure. Unless explicitly stated otherwise in context, singular expressions include plural meanings. In this application, the term “comprise” or “have” is intended to specify the presence of a features, numbers, steps, operations, components, parts, or combinations thereof described in the specification, but should not be construed as precluding the possibility of the presence or addition of one or more other features, numbers, steps, operations, components, parts, or combinations thereof.

Unless otherwise defined, all terms used herein, including technical or scientific terms, have the same meaning as generally understood by those of ordinary skill in the art to which the present disclosure belongs. Terms that are defined in commonly used dictionaries should be interpreted as having a meaning consistent with the meaning they have in the context of the relevant technology, and should not be construed in an idealized or overly formal sense unless explicitly defined in this application.

In addition, when describing with reference to the attached drawings, the same components are given the same reference numerals regardless of the drawing numbers, and redundant descriptions thereof are omitted. In describing the present disclosure, when it is determined that a specific description of a related known technology may unnecessarily obscure the gist of the present disclosure, a detailed description thereof is omitted.

The present disclosure relates to a method of using a fiducial marker using an anatomical structure in a surgical navigation system.

In the present disclosure, the subject performing the method of using a fiducial marker may be referred to as a surgical navigation system or may be a control unit or a processor that overall controls the surgical navigation system. That is, the method of using a fiducial marker of the present disclosure may be composed of an algorithm, which is a type of software, and this algorithm may be executed in a surgical navigation system, a control unit, or a processor that performs the method of using a fiducial marker.

In the present disclosure, 3D reconstruction refers to technology for converting a 2D image into a 3D image. In addition, registration is technology for converting different data sets into a single coordinate system.

A fiducial marker is a marker capable of serving as a reference in medical images. An anatomical structure refers to anatomical structures, and an anatomical landmark refers to an anatomical structure used as a fiducial marker in surgical navigation. A surgical target refers to a specific area or tissue to be operated on, and hard tissue refers to dense tissues such as bones or cartilages.

The present disclosure relates to a method of using a fiducial marker in a surgical navigation system, including a CT process of acquiring a CT image by performing a computed tomography (CT) scan on a patient, CT image-based 3D reconstruction of generating a CT 3D model by using 3D reconstruction technology, based on the CT image, fluorescence process of generating a fluorescent image by capturing, using multiple cameras, an image of an anatomical landmark in which an anatomical structure near a surgical target of the patient is highlighted with a fluorescent contrast agent, multi-camera-based 3D reconstruction of generating a fluorescent 3D model from the fluorescent image by using 3D reconstruction technology, and registration of registering the CT 3D model and the fluorescent 3D model into one model.

The CT process may include performing a computed tomography (CT) scan of the patient including the fiducial marker attached to the patient's skin, and segmenting a CT image captured via the CT scan, by using a selected surgical target and anatomical structure of the patient.

In the CT image-based 3D reconstruction, the CT image may be reconstructed into a 3D model by using marching cube algorithm-based 3D reconstruction technology.

The fluorescence process may include waiting until the fluorescent contrast agent injected to target the anatomical structure is deposited, and capturing an image by using multiple near-infrared cameras when the fluorescent contrast agent is deposited.

The registration may include coarse registration of registering the CT 3D model and the fluorescent 3D model into one model, and fine registration of more precisely registering the model obtained after performing the coarse registration.

FIG. 4 is a flow chart showing a method of using a fiducial marker using an anatomical structure in a surgical navigation system according to an embodiment of the present disclosure.

Referring to FIG. 4, the method includes a CT process (S110) of acquiring a CT image via a computed tomography (CT) scan of a patient, CT image-based 3D reconstruction (S120) of generating a CT 3D model by using 3D reconstruction technology, based on the CT image, a fluorescence process (S130) of generating a fluorescent image by capturing, using multiple cameras, an image of an anatomical landmark in which an anatomical structure near a surgical target of the patient is highlighted with a fluorescent contrast agent, multi-camera-based 3D reconstruction (S140) of generating a fluorescent 3D model from the fluorescent image by using 3D reconstruction technology, coarse registration (S150) of registering the CT 3D model and the fluorescent 3D model into one model, and fine registration (S160) of more precisely registering the model obtained after performing the coarse registration.

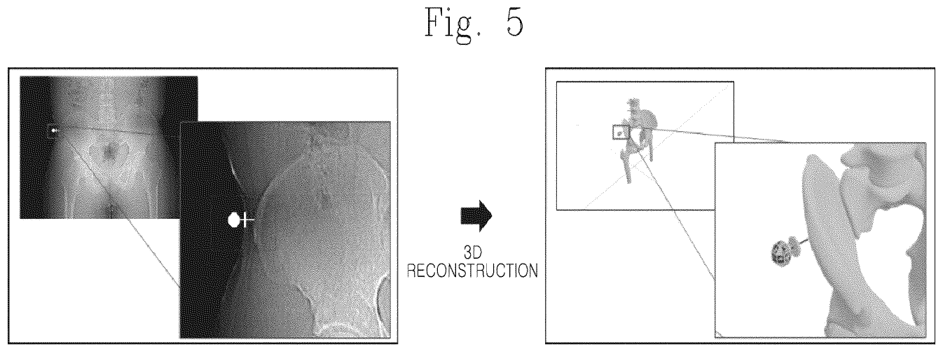

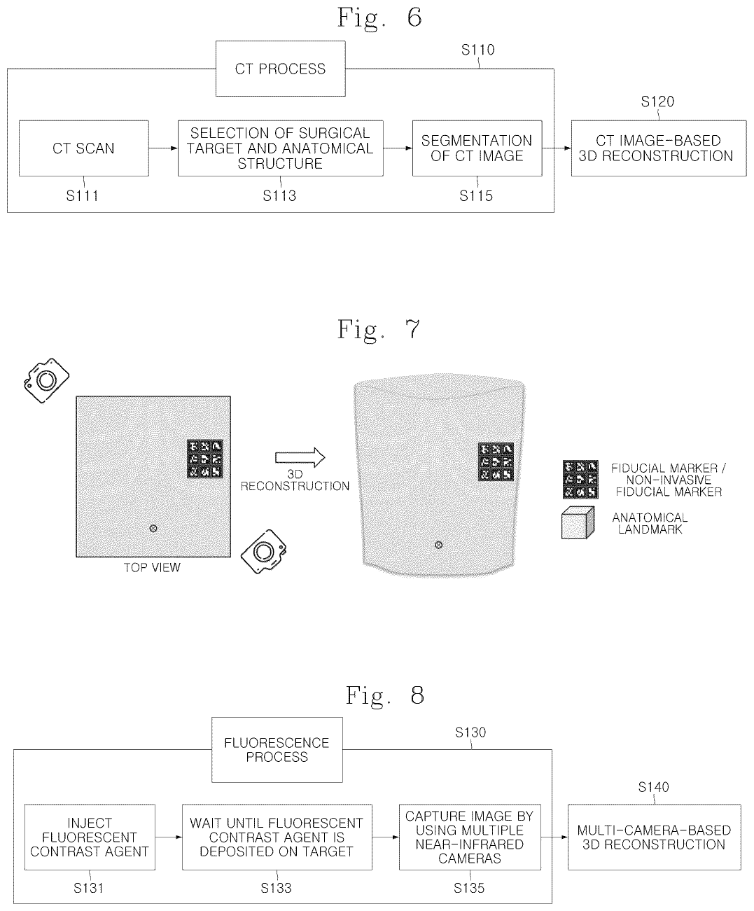

FIG. 5 illustrates 3D reconstruction of a CT image according to an embodiment of the present disclosure, and FIG. 6 is a flowchart illustrating a CT 3D reconstruction process according to an embodiment of the present disclosure.

Referring to FIG. 5 and FIG. 6, the CT process (S110) includes attaching a fiducial marker to the patient's skin and then performing a computed tomography (CT) scan (S111), selecting a surgical target and an anatomical structure (S113), and segmenting a CT image captured via the CT scan (S115).

In an embodiment of the present disclosure, in the CT image-based 3D reconstruction (S120), the CT image may be reconstructed into a 3D model by using marching cube algorithm-based 3D reconstruction technology.

In FIG. 5, the left image is a CT image on which a fiducial marker is displayed, and the right image is a 3D reconstruction image based on a CT image, which is a 3D reconstruction image using a marching cube algorithm.

In the present disclosure, the fluorescence process (S130) and the multi-camera-based 3D reconstruction (S140) are processes of highlighting an anatomical structure with a fluorescent contrast agent and generating a 3D model by using multiple view geometry.

In the present disclosure, an anatomical landmark is a new fiducial marker selected to overlay a 3D model on surgical navigation, based on an anatomical structure that has been segmented from a CT image in advance.

In the present disclosure, an anatomical landmark may be utilized by injecting a fluorescent contrast agent to target an anatomical structure. Here, the contrast agent refers to a drug that increases the contrast of images during imaging diagnostic tests and helps to clearly distinguish tissues or surgical targets.

Since the anatomical landmark is an anatomical structure, the location of which remains unchanged even during open surgery, the anatomical landmark is suitable for serving as a fiducial marker inside the body. In addition, since the anatomical landmark directly utilizes the patient's body, the physical burden is relatively low.

In an embodiment of the present disclosure, the fluorescence process (S130) and the multi-camera-based 3D reconstruction (S140) are processes of capturing an image of an anatomical landmark by using multiple near-infrared cameras and then performing 3D reconstruction. At this time, multiple images acquired through the multiple near-infrared cameras may be 3D reconstructed using multiple view geometry.

FIG. 7 illustrates a fluorescent 3D reconstruction process according to an embodiment of the present disclosure.

FIG. 7 illustrates anatomical landmarks and non-invasive fiducial markers, and the process of reconstructing multiple images acquired through multiple near-infrared cameras into 3D by using multiple view geometry.

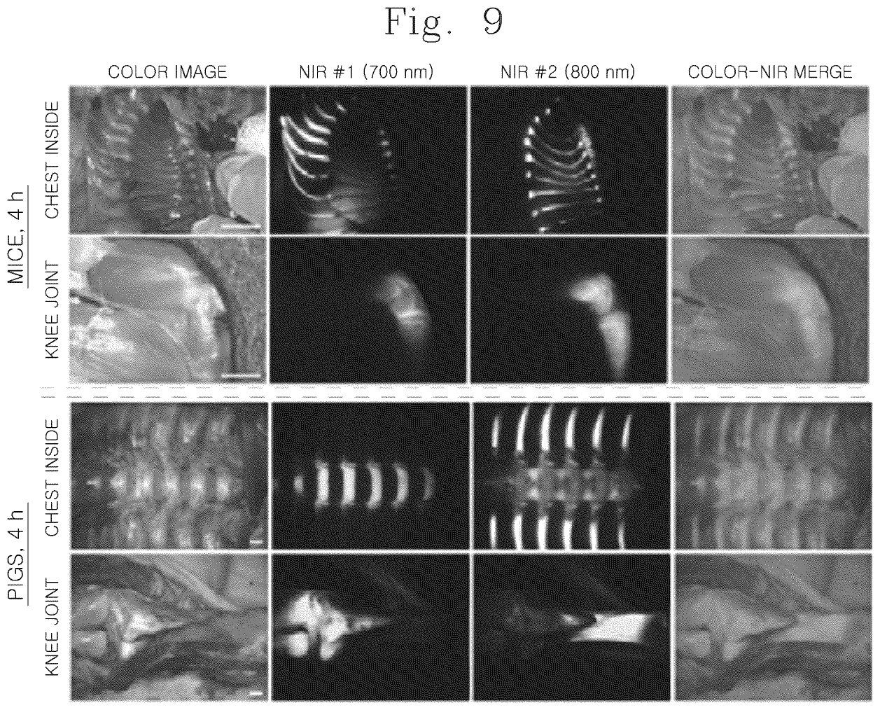

FIG. 8 is a flow chart showing a fluorescent 3D reconstruction process according to an embodiment of the present disclosure.

Referring to FIG. 8, the fluorescence process (S130) includes injecting a fluorescent contrast agent to target an anatomical structure (S131), waiting until the fluorescent contrast agent is deposited on the target (S133), and capturing an image by using multiple near-infrared cameras when the fluorescent contrast agent is deposited on the target (S135).

FIG. 9 is a photograph obtained by using a fluorescent contrast agent.

Referring to FIG. 9, the fluorescent contrast agent in the present disclosure is a contrast agent capable of targeting any tissue in the body and highlighting it on a near-infrared image. By using such a fluorescent contrast agent, an image in which the target is highlighted may be obtained by emitting light of a specific wavelength and simultaneously capturing an image by using a near-infrared camera.

In the present disclosure, the coarse registration (S150) and the fine registration (S160) are processes of registering a CT-based 3D model and a fluorescent 3D model. At this time, the registration result determines the quality of the final 3D model.

In the coarse registration (S150), the CT-based 3D model and the fluorescent 3D model are registered first.

Then, in the fine registration (S160), the model obtained after the coarse registration (S150) is more finely registered using the iterative closest point (ICP) technology.

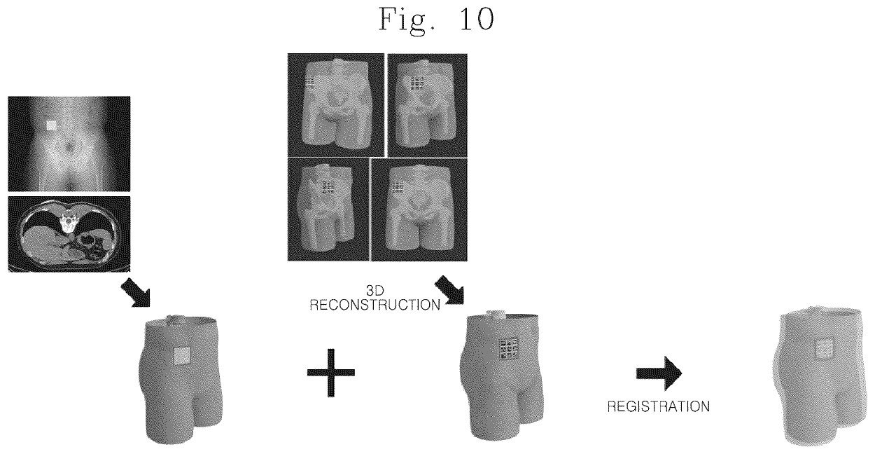

FIG. 10 illustrates a coarse registration process according to an embodiment of the present disclosure.

Referring to FIG. 10, the coarse registration (S150) is a process of merging a CT-based 3D model and a fluorescent 3D model into one model, wherein the CT-based 3D model and the fluorescent 3D model are registered into one model by using a non-invasive fiducial marker as a fiducial point.

FIG. 11 illustrates a registration process to which an ICP algorithm is applied.

As shown in FIG. 11, ICP is technology for registration performed by repeatedly reducing the distance between points that constitute a model.

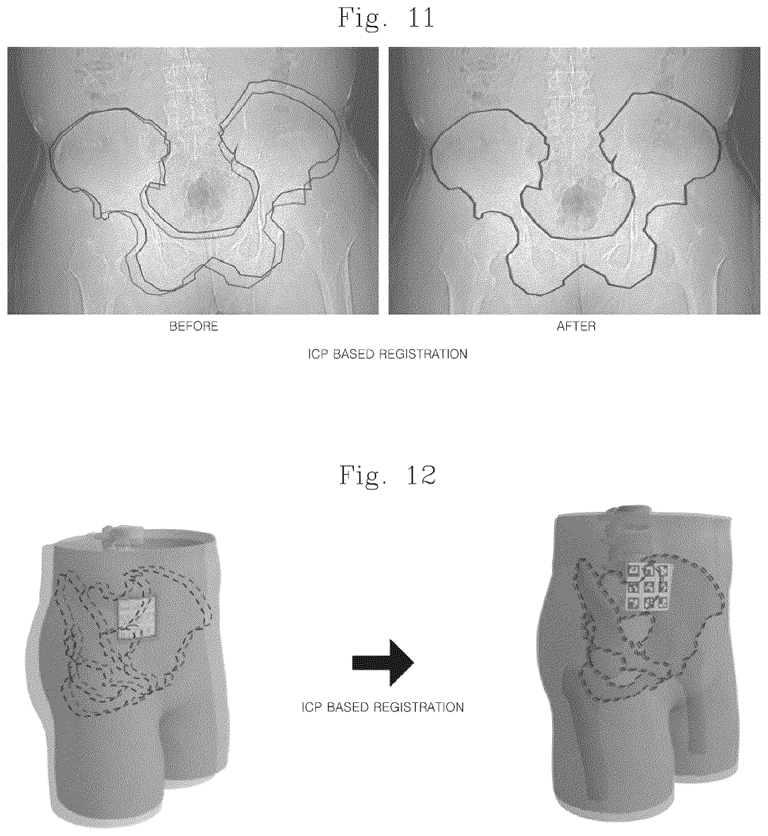

FIG. 12 shows images before and after ICP algorithm technology is applied. In FIG. 12, (a) is an image before application of the ICP algorithm technology, and (b) is an image after application of the ICP algorithm technology.

Referring to FIG. 12, in the present disclosure, the fine registration (S160) is a process of performing more fine registration by using the iterative closest point (ICP) technology on the model obtained after the coarse registration (S150). In an embodiment of the present disclosure, the fine registration (S160) may be performed using the G-ICP technology of the point-to-plane method or the ICP technology of the linear optimization method.

While the present disclosure has been described above using several preferred embodiments, these embodiments are illustrative and not restrictive. Those skilled in the art to which the present disclosure pertains will understand that various changes and modifications may be made without departing from the spirit of the present disclosure and the scope of the claims set forth in the appended claims.

1. A method of using a fiducial marker in a surgical navigation system, the method comprising:
a computed tomography (CT) process for obtaining a CT image via a CT scan of a patient;
CT image-based 3D reconstruction for generating, based on the CT image, a CT 3D model by using 3D reconstruction technology;
a fluorescence process for generating a fluorescent image by capturing, using multiple cameras, an image of an anatomical landmark in which an anatomical structure near a surgical target of the patient is highlighted with a fluorescent contrast agent;
multi-camera-based 3D reconstruction for generating a fluorescent 3D model from the fluorescent image by using 3D reconstruction technology; and
registration for registering the CT 3D model and the fluorescent 3D model into one model.

2. The method of claim 1, wherein the CT process comprises:
performing a computed tomography (CT) scan of the patient including the fiducial marker attached to the patient's skin; and
segmenting the CT image captured via the CT scan, by using a selected surgical target and anatomical structure of the patient.

3. The method of claim 1, wherein the CT image-based 3D reconstruction comprises reconstructing the CT image into a 3D model by using marching cube algorithm-based 3D reconstruction technology.

4. The method of claim 1, wherein the fluorescence process comprises:
waiting until the fluorescent contrast agent injected to target the anatomical structure is deposited; and
capturing an image using multiple near-infrared cameras when the fluorescent contrast agent is deposited.

5. The method of claim 1, wherein the registration comprises:
coarse registration of registering the CT 3D model and the fluorescent 3D model into one model; and
fine registration of more precisely registering the model obtained after performing the coarse registration.
